(19) United States
(12) Patent Application Publication
(10) Pub. No.: US 2008/0234603 A1
(43) Pub. Date: Sep. 25, 2008

(54) ELECTRODE DOME AND METHOD OF USE

(75) Inventors: Gregory J. Bakos, Mason, OH (US); Gary L. Long, Cincinnati, OH (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: Ethicon Endo-Surgery, Inc., Cincinnati, OH (US)

(21) Appl. No.: 11/687,702

(22) Filed: Mar. 19, 2007

(51) Int. Cl. A61B 10/00

(52) U.S. Cl. 600/567

(57) ABSTRACT

Methods and devices are provided for dissecting tissue. In one embodiment, an electro-surgical device is provided having a housing with a hollow interior adapted to receive tissue, such as an organ. The housing can be adapted to couple to a pressure source for suctioning tissue into the housing, and to an energy source for delivering energy to the tissue. The various elements of the device can be adapted such that the initially dispersed energy is passed through the tissue and is concentrated at a location, e.g., connective tissue, having a surface area that is less than a surface area of the electrode, thereby allowing the connective tissue to be dissected. Exemplary methods for dissecting tissue are also provided.

### FIELD OF THE INVENTION

The present invention relates to methods and devices for dissecting tissue.

### BACKGROUND OF THE INVENTION

During natural orifice surgery, it can be difficult to contact and maneuver a targeted organ. When targeting the gall bladder, it can be particularly difficult to expose the connective tissue interface between the gall bladder and an associated liver bed by instruments delivered via an endoscope. Furthermore, precise dissection around the gall bladder is an extremely sensitive and difficult procedure. Damage to the gall bladder and/or liver can frequently occur when using current devices, such as a needle knife, because of the risk of over penetration, or imprecise control at the end of an endoscope that has been maneuvered to target the gall bladder through a natural orifice.

As such, there is a need in the art for devices and methods capable of dissecting tissue.

### SUMMARY OF THE INVENTION

The present invention generally provides methods and devices for dissecting tissue. In one embodiment, an electro-surgical device is provided having a housing with a rim adapted to be positioned against a tissue surface and a hollow interior adapted to receive tissue therein. The housing can be adapted to couple to a pressure source that is effective to apply a suction force to the hollow interior to draw tissue therein. An electrode can be disposed within the hollow interior of the housing and it can be adapted to deliver ablative energy to tissue drawn into the housing to dissect the tissue. The device can also include an elongate tube extending from the housing and coupled to the pressure source, and an electrical connector having a distal end coupled to the electrode and a proximal end adapted to couple to an energy source.

While the shape of the housing can vary, in an exemplary embodiment the housing is dome-shaped. The housing can also be formed from various materials, but it is preferably formed from a non-conductive material. The position of the electrode within the housing can also vary, but in one embodiment the electrode is disposed around an inner surface of the housing. For example, the electrode can be disposed adjacent to the rim of the housing. In certain exemplary embodiments, the electrode can be in the form of a coating disposed on at least a portion of an inner wall of the housing.

In another embodiment, a method for dissecting an organ is provided and includes positioning a housing on an organ. In one embodiment, the housing can be delivered through an endoscope. The method can also include applying a suction force to the housing to draw at least a portion of the organ into the housing, and delivering energy to the housing to dissect at least a portion of connective tissue coupled to the organ. In an exemplary embodiment, energy can be delivered through an electrode disposed within the housing. The electrode preferably has a surface area that is greater than a surface area of the connective tissue being dissected. In certain exemplary embodiments, the organ can be the gall bladder, and the connective tissue is the tissue that extends between the gall bladder and liver such that delivering energy is effective to separate the gall bladder from the liver. The connective tissue can be partially cut with a cutting element prior to delivering energy to facilitate dissection. When energy is delivered, the energy can pass through the organ and connective tissue to a return electrode positioned at a location remote from the organ. The method can also include applying tension to the housing as energy is delivered to pull the organ away from the connective tissue.

### BRIEF DESCRIPTION OF THE DRAWINGS

The invention will be more fully understood from the following detailed description taken in conjunction with the accompanying drawings, in which:

FIG. 1 is a side view of one embodiment of an electro-surgical device;

FIG. 2 is a cross-sectional view of the device of FIG. 1;

FIG. 3A is a perspective view of the device of FIG. 1 showing energy being delivered to a relatively large area of connective tissue extending from the organ;

FIG. 3B is a perspective view of the device of FIG. 1 showing energy being delivered through an organ to a relatively small area of connective tissue extending from the organ;

FIG. 4A is a partially cross-sectional view of the electro-surgical device of FIG. 1 being loaded into an endoscope;

FIG. 4B is a partially cross-sectional view of the embodiment of FIG. 4A showing the electro-surgical device extending from the endoscope;

FIG. 5A is a partially cross-sectional view showing an over-tube with an endoscope disposed therein being positioned adjacent to a treatment area;

FIG. 5B is a partially cross-sectional view of the embodiment of FIG. 5A showing an electro-surgical device being delivered through the endoscope to the treatment area;

FIG. 5C is a partially cross-sectional view of the embodiment of FIG. 5B showing an organ being suctioned into the electro-surgical device;

FIG. 5D is a partially cross-sectional view of the embodiment of FIG. 5C showing connective tissue being dissected; and

FIG. 5E is a partially cross-sectional view of the embodiment of FIG. 5D showing the electro-surgical device and organ being withdrawn from the treatment area.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

In general, methods and devices are provided for dissecting tissue. The methods and devices utilize an ablative energy that is delivered through a relatively large surface area of an organ or other tissue mass and is concentrated in connective tissue, thereby dissecting the connective tissue to detach the organ or other tissue mass. As will be discussed below, the device can be adapted in various ways in order to enhance the therapeutic effect of the device. For example, the device can be sized and shaped to be specifically adapted to receive at least a portion of a desired organ. Additionally, the device can be capable of applying a suction force to the organ to allow the organ to be manipulated and/or to increase the surface area of the organ in communication with an electrode disposed within the device. The electrode can have a surface area and orientation relative to the organ such that energy is initially dispersed over a large surface area of the organ but is concentrated at a second location of relatively smaller surface area as the energy propagates to a return electrode. In an exemplary embodiment, energy is concentrated at a connective tissue which extends between the organ (e.g., gall bladder) and a second biological body (e.g., liver) such that the energy ablates the connective tissue while minimizing damage to any surrounding healthy tissue.

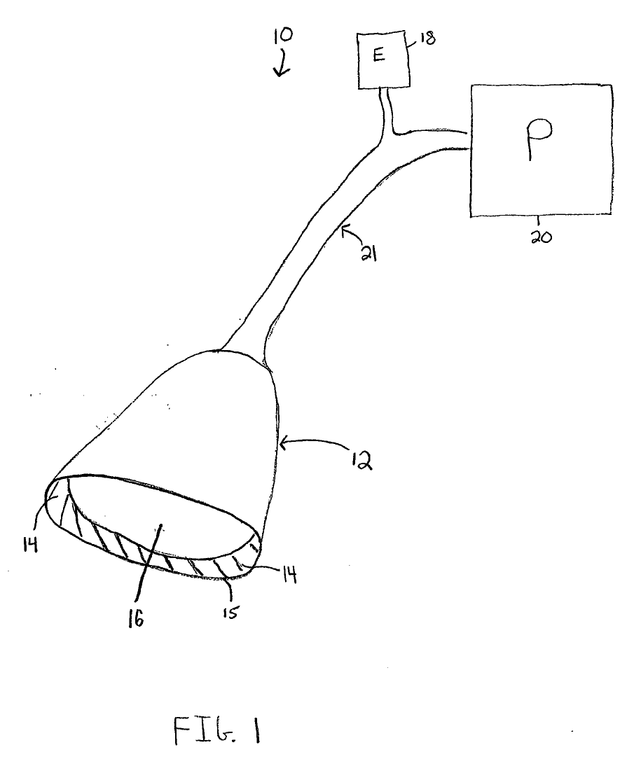

FIG. 1 is an overview of one exemplary embodiment of an electro-surgical device 10. As shown, the device 10 can include a housing adapted to receive tissue, such as an organ. In the illustrated embodiment, the housing is in the form of a dome-shaped element 12. The dome-shaped element 12 can have any shape or configuration capable of receiving at least a portion of an organ or other tissue mass within a hollow interior of the element 12. As will be discussed below, the configuration of the dome-shaped element 12 can allow for a large portion of an inner tissue-facing surface 16 of the element 12 to be placed adjacent to or in direct contact with a large surface area of an organ. The device 10 can also include a pressure source 20 coupled to the dome-shaped element 12 via a suction hose 21 (or other similar device) for applying a suction force to draw an organ into the hollow interior of the dome-shaped element 12. The suction force can be manipulated in order to increase the surface area of the organ in communication with the inner wall 16 of the dome-shaped element 12. The device can further include an electrode 14 in communication with an energy source 18 and disposed within the element. In the illustrated embodiment, the electrode 14 is disposed on the inner wall 16 of the dome-shaped element 12. As will be discussed in detail below, the electrode 14 can be sized and positioned such that the electrode 14 is in electrical communication with a relatively large surface area of the organ and thereby capable of dispersing energy over a large area of the organ. Each of these elements will be described in detail below.

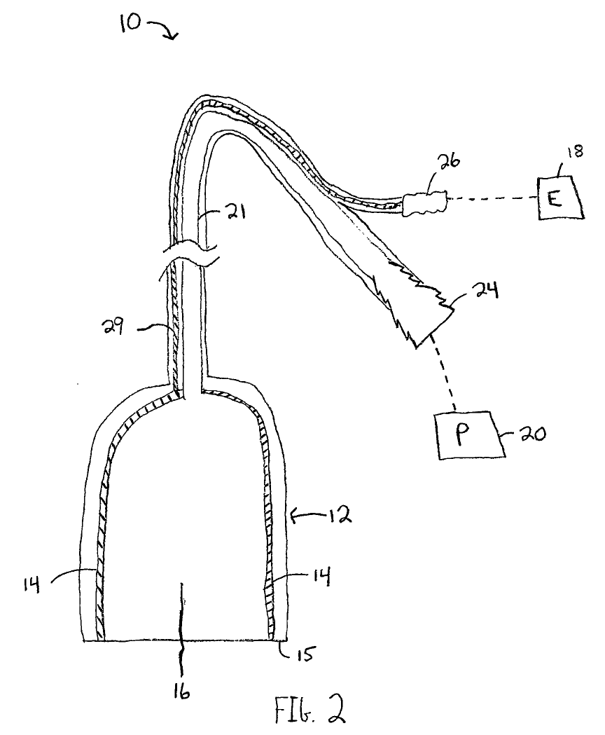

FIG. 2 illustrates the dome-shaped element 12 in more detail. As shown, the dome-shaped element 12 can include a substantially circular outer rim 15, and an inner tissue-facing surface 16 formed within a hollow interior of the element 12. The hollow interior can be sized and configured to receive at least a portion of an organ. As such, the dimensions and shape of the element 12 can be adapted depending on the nature of the procedure. For example, the dome-shaped element 12 can be configured based on the characteristics of the delivery site (i.e., shape and/or type of organ). Further, the element 12 can be adapted to allow for a path or mode delivery. For example, the dome-shaped element 12 can be adapted for delivery to the treatment site via an endoscope.

The dome-shaped element 12 can be formed from a wide range of bio-compatible materials. For example, the element 12 can be formed from a metal or metal alloy, various polymers or polymer mixtures, or combinations of both. Additionally, the element 12 can be formed from a non-conductive material or it can include a non-conductive coating disposed over at least a portion of the element 12. As will be appreciated by those skilled in the art, any such bio-compatible material is within the spirit and scope of the present invention.

As previously indicated, the dome-shaped element 12 can also be configured to couple to a pressure source to allow tissue to be suctioned therein. As shown in FIG. 2, a pressure source 20 is coupled to the dome-shaped element 12 via a suction hose 21 that is in communication with the hollow interior of the element 12. Various techniques can be used to mate the suction hose 21 to a pressure source 20. FIG. 2 illustrates a hose connector 24 formed on a proximal end of the suction hose 21. The pressure source 20, as will be appreciated by those skilled in the art, can be any such source capable of delivering a desired suction force to the element 12 for a desired duration.

In use, the suction force can be applied to tissue, e.g., an organ, positioned adjacent to or partially within the hollow interior of the dome-shaped element 12 so as to draw (or further draw) the organ into the recess. Also, the force can be applied in order to increase the area of the organ in communication with the inner wall 16 of the element 12. Further, the suction force can be adapted so as to allow the organ to be retained within the dome-shaped element 12 as the element 12 applies a mechanical force to the organ, e.g., a pulling and/or twisting force applied to the organ so as to manipulate the organ and facilitate dissection of the organ.

As further shown in FIG. 2, in order to deliver energy to an organ suctioned into the element 12, an electrode 14 can be disposed with the hollow interior of the dome-shaped element 12. In an exemplary embodiment, the electrode 14 is formed or disposed on the inner tissue facing surface 16 of the dome-shaped element 12. As further shown in FIG. 2, the electrode 14 can be in electrical communication with an energy source, generally indicated by element 18. In the exemplary embodiment, an electrical wire 29 extends through or along the suction hose 21 and has a distal end that couples the electrode 14 and a proximal end with an electrical connector 26 for mating to an energy source. As appreciated by those skilled in the art, the electrical connector 26 can be coupled to the energy source 18 using any number of mechanisms (e.g., a power cord). Regarding the energy source 18, those skilled in the art will appreciate that any type of energy source 18 capable of delivering a desired amount of energy to the electrode 14 for a desired duration is within the spirit and scope of the present invention.

The electrode 14 can be disposed within the dome-shaped element 12 using any method known in the art. For example, the electrode 14 can be a surface coating applied to a desired area of the inner wall 16. Alternatively, a desired portion of the inner wall 16 can function as the electrode 14. The electrode 14 can also be formed from any type of conductive material. In an exemplary embodiment, the electrode 14 is formed from a plated metal. Those skilled in the art will appreciate that any such method of coupling or forming any type of conductive material to the dome-shaped element 12 is within the spirit and scope of the present invention. The electrode 14 can also be positioned at various areas within the dome-shaped element 12. For example, the electrode 14 can be positioned adjacent to at least a portion of the outer rim 15 of the dome-shaped element 12. Alternatively, the electrode 14 can cover substantially the entire surface of the inner wall 16 of the dome-shaped element 12.

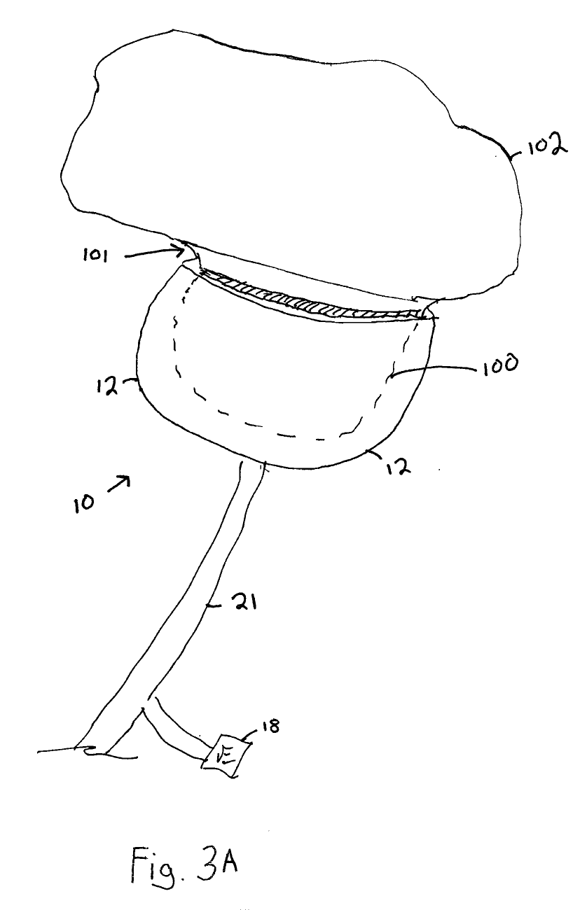

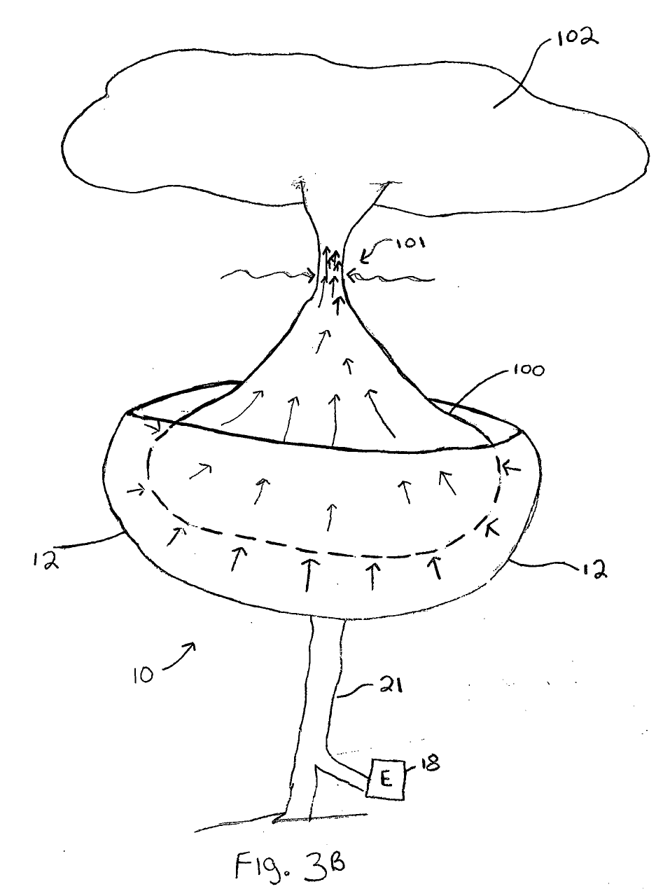

FIG. 3A shows an example of the dome-shaped element 12 contacting a first organ (e.g., gallbladder) which is engaged to a second organ (e.g., liver) by a relatively large surface area of connective tissue 101. The energy density at the edge of the electrode 14 can be adapted to be large relative to the entire surface of the electrode 14 such that heating (due to an increase in current density) of the tissue (indicated in FIG. 3A by a hashed stripe) can take place. This heating can be optimized to weaken the tissue 101 to a point where the dome-shaped element 12 can peel the tissue 101 from the second organ (liver) 102. In such an embodiment, when the first organ 100 is pulled a substantial amount away from the second organ 102, the amount of connective tissue 101 can be decreased.

FIG. 3B shows an example of the device 10 in use wherein energy is delivered over a large area of the organ 100 and concentrated at a second location to produce a therapeutic effect (i.e., ablation of tissue). In this example, the device 10 is depicted as delivering energy to an organ (e.g., a gall bladder) 100 that is connected to a second biological body (e.g., the liver) 102 by a connective tissue 101. The surface area of tissue 101 can be decreased (thereby increasing ablation efficiency) by a number of methods, for example by utilizing the steps described above in relation to FIG. 3A. As shown, a portion of the organ is suctioned into the hollow interior of the dome shaped element 12 using a suction force delivered via the suction hose 21. This increases the surface area of the organ that is in communication with the electrode 14. Once the organ is placed into electrical communication with the electrode 14, energy (as represented by arrows) can be delivered to the electrode, whereby it will be dispersed over a relatively large area of the organ. The large area surface of the organ, as well as the amount of energy being delivered, can result in a non-ablative energy being delivered to the organ. As the energy propagates to a return electrode (not shown) located at a remote position, the energy will pass through the connective tissue 101. Due to the relatively small surface area of the connective tissue 101 as compared to the initial area of delivery, the energy will ablate the connective tissue 101 thereby allowing for removal of the organ from the treatment area. In an exemplary embodiment, the connective tissue has a surface area that is less than a surface area of the electrode. This can optionally be achieved by partially cutting the connective tissue prior to delivering energy, and/or pulling the organ to stretch the connective tissue prior to delivering energy.

The electro-surgical device 10 can be delivered to the treatment area using various methods. In one exemplary embodiment, the device can be delivered endoscopically to the treatment area, as shown in FIGS. 4A and 4B. As shown in FIG. 4A, the electro-surgical device 10 can be loaded into a distal end of an endoscope 30 such that the dome-shaped element 12 can reside adjacent to or within a distal opening of the endoscope 30, and a proximal end of the device can extend from a proximal end, e.g. a side-channel 32, of the endoscope 30. To facilitate delivery using an endoscope, the dome-shaped element 12 can optionally be configured to collapse to allow it to be received within the endoscope. This can be achieved by, for example, by removing the electrical connector 26 and hose connector 24 thereby allowing the proximal end of the suction tube to be inserted into a distal end of the endoscope 30. Once the tube exits the side channel 32 of the scope 30, the electrical connector 26 and hose connector 24 can be reattached for use. FIG. 4B shows the device 10 positioned within the endoscope 30. Those skilled in the art will appreciate that a wide range of endoscopes known in the art can be used with the present invention.

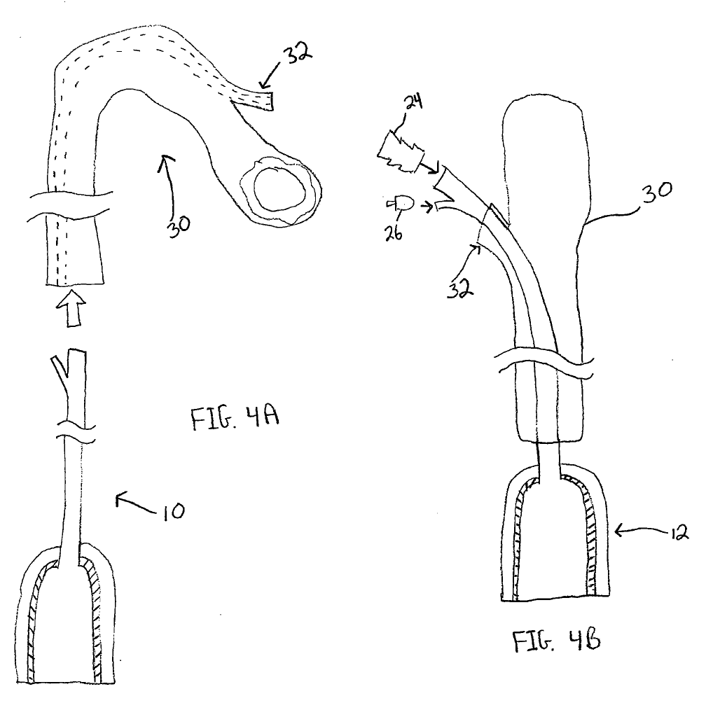

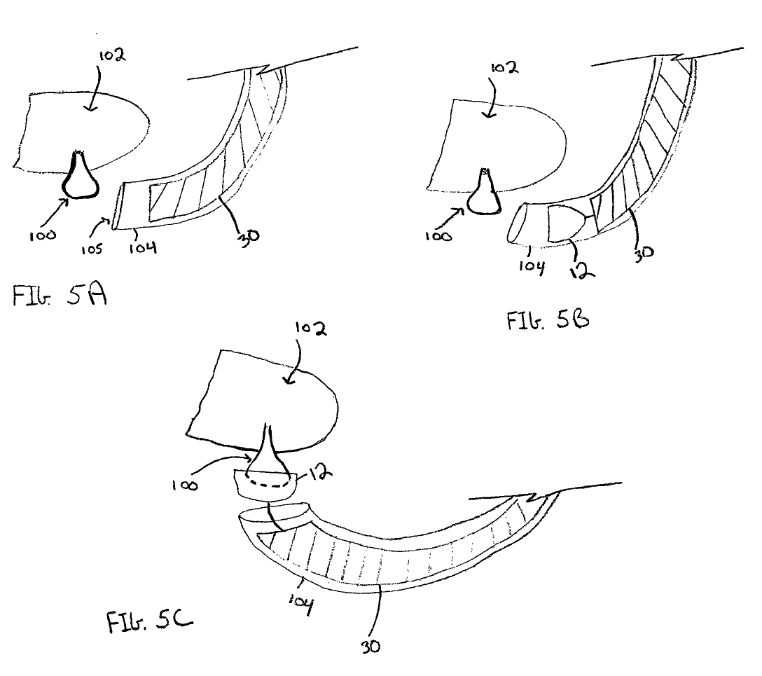

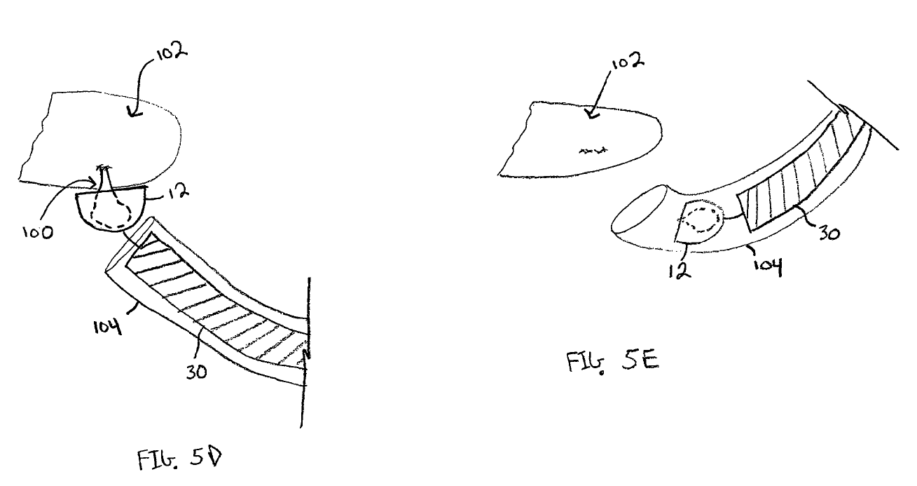

FIGS. 5A-5E provide an illustrative example of the device 10 in use. In this example, the device 10 is being utilized to dissect a gall bladder 100 connected to a liver 102 by a connective tissue. The liver has certain characteristics capable of facilitating use of the presently disclosed device. For example, the liver, being a blood filter, has a great ability to dispense heat energy due to the high volume of blood flow. As will be appreciated by those skilled in the art, the device can be used to perform a wide range of procedures. The following example is merely illustrative.

As shown in FIG. 5A, the endoscope 30 can optionally be disposed within an over-tube 104 and delivered to the treatment area. The duct and artery extending from the gall bladder can optionally be cut using a cutting device disposed through the endoscope 30. Once the distal end 105 of the over-tube 104 is positioned adjacent the organ 100, the scope 30 can be withdrawn in order to load the electro-surgical device 10 into the scope 30. The scope 30, with the device 10 disposed therein, can then be reintroduced into the over-tube 104. FIG. 5B shows the dome-shaped element 12 protruding from a distal end of the endoscope 30 as the endoscope 30 is advanced to the treatment site via the previously positioned over-tube 104. As shown in FIG. 5C, the endoscope 30 can advance along the over-tube 104 thereby allowing the dome-shaped element 12 to exit the over-tube 104 and engage at least a portion of the organ 100.

Once the dome-shaped element 12 is placed adjacent to or in contact with the organ, the suction force and/or energy, as shown in FIG. 5D and discussed above, can be applied to the organ 100 in order to draw the organ into the recess and ablate the connective tissue (as discussed above). In one embodiment, a cutting element (e.g., a monopolar cutting device) can be utilized to further reduce the surface area of the connective tissue thereby allowing for a higher concentration of energy at the connective tissue. These various mechanisms (application of energy, mechanical force, and/or use of a cutting element) can be utilized in any order and/or combination until the connective tissue has been ablated and the organ may be removed (see FIG. 5E).

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that device is sterilized. This can be done by any number of ways known to those skilled in the art including beta or gamma radiation, ethylene oxide, steam.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. An electro-surgical device, comprising:
a housing having a rim adapted to be positioned against a tissue surface and a hollow interior adapted to receive tissue therein;
a pressure source in communication with the housing and adapted to apply a suction force to the hollow interior to draw tissue therein; and
an electrode disposed within the hollow interior of the housing and adapted to deliver ablative energy to tissue drawn into the housing to dissect the tissue.

2. The electro-surgical device of claim 1, further comprising an elongate tube extending from the housing and coupled to the pressure source.

3. The electro-surgical device of claim 1, further comprising an electrical connector having a distal end coupled to the electrode and a proximal end adapted to couple to an energy source.

4. The electro-surgical device of claim 1, wherein the housing is dome-shaped.

5. The electro-surgical device of claim 1, wherein the housing is formed from a non-conductive material.

6. The electro-surgical device of claim 1, wherein the electrode is disposed around an inner surface of the housing.

7. The electro-surgical device of claim 6, wherein the electrode is disposed adjacent to the rim of the housing.

8. The electro-surgical device of claim 1, wherein the electrode comprises a coating disposed on at least a portion of an inner wall of the housing.

9. An electro-surgical device, comprising:
a dome-shaped housing adapted to couple to a pressure source such that tissue can be suctioned into the dome-shaped housing; and
an electrode disposed around an inner wall of the dome-shaped housing and adapted to couple to an energy source such that ablative energy can be delivered to tissue suctioned into the dome-shaped housing.

10. The electro-surgical device of claim 9, further comprising an elongate tube extending from the housing and coupled to a pressure source.

11. The electro-surgical device of claim 9, further comprising an electrical connector having a distal end coupled to the electrode and a proximal end adapted to couple to an energy source.

12. The electro-surgical device of claim 9, wherein the housing is formed from a non-conductive material.

13. The electro-surgical device of claim 9, wherein the electrode is disposed adjacent to a tissue-contacting rim of the housing.

14. The electro-surgical device of claim 9, wherein the electrode comprises a coating disposed around the inner wall of the housing.

15. A method for dissecting an organ, comprising:
positioning a housing on an organ;
applying a suction force to the housing to draw at least a portion of the organ into the housing; and
delivering energy to the housing to dissect at least a portion of connective tissue coupled to the organ.

16. The method of claim 15, wherein energy is delivered through an electrode disposed within the housing.

17. The method of claim 16, wherein the electrode has a surface area that is greater than a surface area of the connective tissue being dissected.

18. The method of claim 15, wherein the organ comprises the gall bladder, and the connective tissue extends between the gall bladder and liver such that delivering energy is effective to separate the gall bladder from the liver.

19. The method of claim 15, further comprising, prior to positioning the housing, introducing the housing through an endoscope disposed through a body lumen.

20. The method of claim 15, further comprising, prior to delivering energy, partially cutting a portion of the connective tissue with a cutting element.

21. The method of claim 15, wherein energy passes through the organ and connective tissue to a return electrode positioned at a location remote from the organ.

22. The method of claim 15, further comprising applying tension to the housing as energy is delivered to pull the organ away from the connective tissue.
